(19) United States
(12) Patent Application Publication
(10) Pub. No.: US 2013/0211072 A1
(43) Pub. Date: Aug. 15, 2013

(54) CAPROLACTAM RECOVERY WITH MEMBRANE TREATMENT

(71) Applicants: Maria Elena Avramescu, Roermond (NL); Johan Thomas Tinge, Sittard (NL)

(72) Inventors: Maria Elena Avramescu, Roermond (NL); Johan Thomas Tinge, Sittard (NL)

(21) Appl. No.: 13/516,296

(22) PCT Filed: Dec. 17, 2010
(86) PCT No.: PCT/EP10/70097

§ 371 Date: Sep. 14, 2012

(30) Foreign Application Priority Data
Dec. 18, 2009 (EP) EP09179903.1

(51) Int. Cl. C07D 223/10

(52) U.S. Cl. CPC C07D 223/10
USPC 540/540

(57) ABSTRACT

The invention relates to a process for treating an aqueous solution containing ε-caprolactam, ammonium sulphate and one or more other impurities, comprising one or more organic impurities from a caprolactam production process and optionally other salts than ammonium sulphate, by means of a membrane process, thereby obtaining a retentate and a permeate, in which process the membrane used is selected from the group of polyether sulphone membranes, sulphonated polyether sulphone membranes, polyester membranes, polysulphone membranes, aromatic polyamide membranes, polyvinyl alcohol membranes, polypiperazine membranes, cellulose acetate membranes, titanium oxide membranes, zirconium oxide membranes and aluminium oxide membranes, having a molecular weight cut off in the range of 100-1000 g/mol; and wherein more than 60 wt. % of the caprolactam in the aqueous solution is passed through a membrane to the permeate side, to obtain a purified caprolactam containing permeate stream, and wherein at least 50 wt. % of the organic impurities are retained in the retentate solution.

The invention relates to a process for treating an aqueous solution containing ε-caprolactam (hereinafter also ‘caprolactam’), ammonium sulphate and one or more other impurities, comprising one or more organic impurities from a caprolactam production process and optionally other salts than ammonium sulphate, by means of a membrane process, thereby obtaining a retentate and a permeate.

Aqueous solutions containing caprolactam may be formed in various processes, such as in processes directed at the purification of crude caprolactam prepared via a Beckmann rearrangement process or another process of preparing caprolactam. Suitable processes for preparing caprolactam are generally known in the art, e.g. from Ullmann's encyclopedia of Industrial Chemistry, for instance the 7^{th} edition (2005).

An aqueous solution comprising caprolactam, such as caprolactam obtained from a conventional Beckmann rearrangement process wherein the Beckmann rearrangement reaction mixture is generally subjected to neutralisation with aqueous ammonia, typically contains ammonium sulphate and one or more other impurities, especially other salts and at least one organic impurity from a caprolactam production process. Typical organic impurities include organic compounds with molecular weight higher than that of caprolactam, and often may be high-boiling organic compounds, in particular sulphonated organic compounds. The term ‘high-boiling’ is used herein for compounds having a higher boiling point than caprolactam. Caprolactam may, for instance, be recovered from this mixture by first using a liquid-liquid extraction step with an organic solvent, for instance benzene. Known extraction processes are e.g. described in Kirk-Othmer Encyclopedia of Chemical Technology, (John Wiley & Sons 4 December 2000; on line), WO 98/49140 and WO 2002/070475.

After extraction, caprolactam is usually recovered from the organic phase obrtained. However, in practice not all caprolactam will be extracted. The aqueous phase from which caprolactam has been extracted, will still contain some caprolactam, usually at least about 0.2 wt. % (in particular 0.2-3 wt. %). This aqueous phase further comprises ammonium sulphate and one or more inorganic and organic impurities. Usually, the ammonium sulphate content is at least about 1 wt. % (in particular 1-5 wt. %). The organic impurity content (for instance the content of sulphonated caprolactam) is usually at least about 0.5 wt. % (in particular 1.0-2.5 wt. %). In a conventional process, only a minor part of the aqueous phase, typically 30 wt. % or less, can be recycled, for instance, via the neutralisation zone into the extraction zone. Otherwise organic impurity (in particular sulphonated organic compounds) would build up in the process, which would result in an unacceptable loss of caprolactam quality (i.e. purity will be insufficient). Therefore, most of the aqueous phase leaving the extraction zone is in general discarded and optionally treated so that organic materials can be burnt in an incinerator.

Having to discard a large aqueous waste volume is undesired, inter alia from an environmental viewpoint. For instance, in case the waste stream is introduced into an incinerator, a large volume of water in the waste results in high energy consumption needed to evaporate the water. Further, the waste stream typically contains ammonium sulphate, which upon incineration can be converted into sulphur dioxide, a severe pollutant causing acid rain. Accordingly, ammonium sulphate preferably is not sent to an incinerator. Moreover, loss of ammonium sulphate is undesired because it is a useful fertiliser.

It is an object of the present invention to provide a novel process for treating an aqueous solution containing ε-caprolactam, ammonium sulphate and one or more other impurities, comprising one or more organic impurities from a caprolactam production process and optionally other salts than ammonium sulphate, by means of a membrane process, thereby obtaining a retentate and a permeate, which novel process can serve as an alternative to known methods.

It is a further object to provide a novel process that is more efficient than the method described above in that it is less energy consuming and/or in that more pure caprolactam is recovered and/or in that more ammonium sulphate is recovered.

One or more objects that may be met in accordance with the invention will become apparent from the description and/or claims herein below.

It has now been found that one or more objects underlying the invention are met by treating the aqueous liquid in a specific manner.

Accordingly, the present invention relates to a process for treating an aqueous solution containing caprolactam, ammonium sulphate and one or more other impurities, comprising one or more organic impurities from a caprolactam production process and optionally other salts than ammonium sulphate, by means of a membrane process, thereby obtaining a retentate and a permeate, wherein the membrane used is selected from the group of polyether sulphone membranes, sulphonated polyether sulphone membranes, polyester membranes, polysulphone membranes, aromatic polyamide membranes, polyvinyl alcohol membranes, polypiperazine membranes, cellulose acetate membranes, titanium oxide membranes, zirconium oxide membranes and aluminium oxide membranes, having a molecular weight cut off in the range of 100-1000 g/mol; and wherein more than 60 wt. % of the caprolactam in the aqueous solution is passed through a membrane to the permeate side, to obtain a purified caprolactam containing permeate stream, and wherein at least 50 wt. % of the organic impurities are retained in the retentate solution.

Preferably, the molecular weight cut off of the membrane used is, within the aforementione range, in the narrower range of 150-400 g/mol.

In this way a caprolactam stream is obtained which effectively is improved in respect of (lowered) impurities content. The impurities, in particular at least the organic impurities are concentrated in the retentate.

The membranes mentioned above for use in the process of the present invention is all belong to the groups of polymeric, composite and inorganic nanofiltration membranes, and have a molecular weight cut off in the range of 100-1000 g/mol.

In accordance with the invention it has, moreover, been found possible to recover considerably more pure caprolactam from aqueous streams otherwise often to be treated as waste streams than in processes of the prior art.

For instance, in the process as described in U.S. Pat. No. 4,634,531 an aqueous caprolactam-containing stream is subjected to reverse osmosis through a membrane, obtaining a permeate stream for removal of water and a retentate in which the caprolactam has been concentrated. The process according to the present invention essentially achieves the opposite.

Another example of a process for treatment of an aqueous stream from a caprolactam plant by means of a membrane process is described in Hu, Yuehua et al. (Huagong Jinzhan, Vol.23, no.10, 2004, pages 1135-1137), wherein also reverse osmosis is used, now subsequent to an ultrafiltration step for pre-filtration, and wherein the caprolactam is mainly recovered via the retentate. The aim of the tratment of said reference is to obtain a purified water stream which may be re-used.

It is further noticed that Van der Brugger B., et al. (Journal of membrane Science, Vol.156, no.1, 1999, pages 29-41), show that various membranes can be used for separation of single components in specific ratios into retentate and permeate solutions, but this document only shows examples where one single component is present in the aqueous solvent, but without providing any teaching as to selectivity of such separation for more complex mixtures, and in particular not for mixtures comprising one or more salts and ammonium sulphate.

The present invention allows a considerable reduction in organic impurities concentration, and in specific embodiments of the invention also in concentration of ammonium sulphate, in the permeate, as compared to the concentrations in the aqueous solution prior to the membrane filtration step.

Further, the present invention allows a considerable reduction in waste streams, as the caprolactam may suitably be recovered from the permeate on an industrial scale, and the amount of the retentate (which may need to be discarded) is typically reduced compared to the amount discarded in the processes according to the prior art. It is envisaged that the invention allows a reduction in waste water (that would be led into an incinerator or otherwise discarded) by a factor 2-10, in particular by a factor 3-5. Thereby, energy savings are obtained.

The term ‘or’ as used herein means ‘and/or’ unless specified otherwise.

The term ‘a’ or ‘an’ as used herein means ‘at least one’ unless specified other wise.

When referring to a ‘noun’ (e.g. a compound, an additive etc.) in singular, the plural is meant to be included, unless specified otherwise.

When referred herein to organic impurities in general, these may in principle be selected from all organic compounds other than caprolactam, and in particular from the group of organic compounds that have been used or have formed in the preparation of caprolactam, or the subsequent treatment of a reaction product comprising caprolactam, or that have been present as impurities in any of the organic compounds or solvents used in such preparation. Organic impurities may be low-boiling organic compounds, i.e. having a lower boiling point than caprolactam (so called ‘lights’ of ‘light compounds’), or high-boiling organic compounds, i.e. having a higher boiling point than caprolactam (so called ‘heavies’ or ‘heavy compounds’).

The process according to the present invention is preferably carried out as a continuous process.

The aqueous solution treated in accordance with the invention contains at least one organic impurity, in particular, at least one sulphur containing organic compound, more in particular sulphonated caprolactam. When referred herein to a concentration of an organic sulphur-containing compound the concentration is based on the weight of elemental sulphur in the compounds, as may be determined by element analysis (for sulphur). For element analysis, inductively coupled plasma-atomic emission spectrometry (ICP-AES) may be used. Before analysis, the sample may be subjected to a micro-wave destruction treatment, after it has been mixed with aqua regia.

The aqueous solution subjected to the membrane treatment step may in principle be any aqueous solution comprising said compounds. In a specific embodiment, the aqueous solution may be obtained from a caprolactam purification process, more in particular it may be a stream that is conventionally considered a waste stream in such process, i.e. a stream that would otherwise be discarded or used for preparing a caprolactam product from which only a lower grade quality of nylon can be prepared. In particular, a method according to the invention may be a method wherein the caprolactam has been prepared from cyclohexanone, for instance, via an oximation process using hydroxylammonium sulphate or hydroxylammonium phosphate followed by a Beckmann rearrangement and neutralisation with aqueous ammonia.

The aqueous liquid to be subjected to the membrane treatment step according to the invention usually comprises at least 0.5 wt. % caprolactam, in particular at least 1.0 wt. % caprolactam, more in particular at least 1.5 wt. % caprolactam. Usually, the caprolactam concentration in the aqueous liquid to be subjected to the membrane treatment step is less than 15 wt. %, preferably 10 wt. % or less, in particular 5 wt. % or less. In a particularly advantageous embodiment, the caprolactam concentration in the aqueous liquid to be subjected to the membrane treatment step according to the invention is 3.5 wt. % or less.

The ammonium sulphate concentration in the aqueous liquid to be subjected to the membrane treatment step according to the invention usually is less than 15 wt. %, in particular 10 wt. % or less. For an advantageous permeate flux, the ammonium sulphate concentration in the aqueous liquid to be subjected to the membrane treatment step is 5 wt. % or less, in particular about 4 wt. % or less. The aqueous liquid to be subjected to the membrane treatment step according to the invention usually comprises at least 0.5 wt. % ammonium sulphate, in particular at least 1.5 wt. % ammonium sulphate, more in particular at least 2.5 wt. % ammonium sulphate.

The total organic impurity concentration in the aqueous liquid to be subjected to the membrane treatment step according to the invention usually is at least of 0.5 wt. %, in particular at least 1.0 wt. %. The total organic impurity concentration in the aqueous liquid to be subjected to this treatment usually is less than 5 wt. %, in particular 3 wt. % or less, more in particular 2 wt. % or less. Generally more than half, in particular more than 4/5 or more than 9/10 thereof will consist of one or more heavies, and in particular in case the caprolactam has been prepared in a Beckmann rearrangement process by one or more sulphur containing organic compounds, such as sulphonated caprolactam. In a specific embodiment, the sulphur containing organic compounds concentration in the aqueous liquid to be subjected to the membrane treatment step according to the invention is in the range of 0.3-4 wt. %, more specifically in the range of 1-2 wt. %.

In the process according to the invention preferably at least 50 wt. %, most preferably at least 60 wt. % of the ammonium sulphate is retained in the retentate solution. If more than 50 wt. % of the caprolactam is passed to the permeate side, and more than 50 wt. % of the ammonium sulphate is retained in the retentate, the ratio caprolactam vs. ammonium sulphate will be improved and higher purity of the caprolactam-containing solution is obtained. This may be called a process of ammonium sulphate reduction relative to caprolactam (i.e. the ratio of the ammonium sulphate content to the content of caprolactam in the solution before the membrane treatment step is higher than the ratio of the ammonium sulphate content to the content of caprolactam in the permeate). Accordingly, the ammonium sulphate reduction relative to caprolactam may be a factor 2 or more, in particular a factor 4-15, more in particular a factor 6-12.

It is, moreover, preferred that at least part of the permeate solution stream is re-used in a caprolactam production process. Most suitably, the permeate solution stream is then combined with streams in the purification section of the caprolactam production process, or in a section of another process with streams containing caprolactam, such as in a nylon-6 production process. In this way losses of caprolactam can be kept at a minimum.

A preferred example of the caprolactam purification process is a process wherein an aqueous stream comprising caprolactam and impurities is subjected to liquid-liquid extraction using an organic phase, thereby forming a second aqueous solution which now is to be treated by the membrane process of the invention. As will be understood by the skilled person, the membrane treatment method according to the invention may be employed in combination with another type of process for the preparation and/or purification of caprolactam.

In a preferred embodiment of the process according to the invention, the caprolactam process from which the aqueous stream to be treated is taken is a hydroxylammonium sulphate or phosphate oxime process with formation of the oxime from cyclohexanone via an oximation process followed by Beckmann rearrangement of the oxime, neutralisation with aqueous ammonia and purification of the caprolactam obtained.

According to the present invention more than 60 wt. % of the caprolactam is passed through the membrane to the permeate side. In particular at least 75 wt. %, preferably at least 80 wt. %, of the caprolactam in the aqueous solution is passed through the membrane to the permeate side.

Accordingly, at least more than 60 wt. %, in particular at least 75 wt. % of the caprolactam in the aqueous stream to be treated in the membrane step may be recovered via the permeate. In practice, the permeate thus may in particular contain about 80% or less of the caprolactam present in the aqueous solution prior to the membrane treatment step.

In a preferred embodiment of the invention, the caprolactam concentration in the permeate stream is higher than the caprolactam concentration in the aqueous solution before the treatment by means of the membrane process.

In a further preferred embodiment the caprolactam concentration in the permeate stream is, in relative terms, at least 1%, preferably at least 3%, more preferably at least 5%, and most preferably at least 10% higher than the caprolactam concentration in the solution that is subjected to the membrane treatment. For instance, if the feed solution contains 2.0 wt. % of caprolactam, then the permeate obtained according to the invention contains at least 2.02 wt. % of caprolactam (which in relative terms means 1% more).

It is specifically preferred that at least 60 wt. %, preferably at least 75 wt. %, of the organic impurities are retained in the retentate solution.

In a very preferred embodiment of the invention the total concentration of organic impurities in the permeate is at least 1.5 times lower, preferably at least 2 times lower, more preferably at least 4 times lower, even more preferably at least 6 times lower, and most preferably at least 10 times lower than their total concentration in the aqueous solution before the treatment by means of the membrane process.

Accordingly, the reduction in organic impurity concentration relative to caprolactam (ceteris paribus defined in the same way as stated above with respect to ammonium sulphate) may be a factor 2 or more, in particular a factor 4-15, more in particular a factor 6-12.

As mentioned above, for the methods according to the invention a nanofiltration membrane process (i.e. a process using at least one nanofiltration membrane) is used having a molecular weight cut off in the range of 100-1000 g/mol. In particular, the molecular weight cut off may be at least 150 g/mol, more in particular at least 200 g/mol. In particular, the molecular weight cut off may be 500 g/mol or less, more in particular 400 g/mol or less, or 350 g/mol or less. The molecular weight cut off as used herein is, in general, the value as specified by the membrane supplier.

In particular, thus, the membrane used is selected from the group of polyether sulphone membranes, sulphonated polyether sulphone membranes, polyester membranes, polysulphone membranes, aromatic polyamide membranes, polyvinyl alcohol membranes, polypiperazine membranes, cellulose acetate membranes, titanium oxide membranes, zirconium oxide membranes and aluminium oxide membranes, having a molecular weight cut off in the range of 100-1000 g/mol, preferably in the range of 150-400 g/mol.

For the membrane treatment of the invention, in particular for the nanofiltration membrane treatment, any nanofiltration equipment may be used. In general, the equipment comprises at least one nanofiltration membrane element, for dividing the feed into a retentate and permeate section. The nanofiltration equipment usually also include one or more pressure controllers and one or more flow controllers, such as pumps, valves, flow meters, pressure meters and the like. The equipment may also include several nanofiltration membrane elements in different combinations, arranged in parallel or series.

Use particularly may be made of a commercially available membrane. Such membranes, in particular nanofiltration membranes are, e.g., available from Dow

Water Solutions (Midland Mich., USA), GE Osmonics (Minnetonka, Minn., USA), Trisep Corporation (Goleta Calif., USA) or Koch Membrane Systems (Wilmington Mass., USA; or Koch, Germany). Polyether sulphone membranes, sulphonated polyether sulphone membranes, polyester membranes, polysulphone membranes, aromatic polyamide membranes, polyvinyl alcohol membranes, polypiperazine membranes and cellulose acetate membranes are examples of polymeric nanofiltration membranes that may be used.

Inorganic, e.g., ceramic membranes can also be used for the nanofiltration. Examples of ceramic nanofiltration membranes that may be used include titanium oxide membranes, zirconium oxide membranes and aluminium oxide membranes.

In particular, for the nanofiltration membrane treatment step at least one nanofiltration membrane may be used selected from the group of polyamide nanofiltration membranes, polysulphone nanofiltration membranes, cellulose acetate nanofiltration membranes, polypiperazineamide nanofiltration membranes, polysulphonated polyethersulphon nanofiltration membranes, and polypropylene nanofiltration membranes. In particular, good results have been achieved with a polyamide nanofiltration membrane.

In particular, a composite membrane made by interfacial polymerisation may be used.

The (nanofiltration) membranes used may have been chemically or physically modified, e.g. to reduce fouling-tendency and/or increasing their flux and/or permeability, and/or increasing their rejection.

The (nanofiltration) membrane may be an ionic membrane, i.e. it may contain ionisable functional groups (typically acid groups or base group groups). The ionisable group can be functional groups that are either cationic or anionic at the pH of the liquid that is to be filtrated. Also, a neutral membrane may be used, such as a membrane that is free of ionisable functional groups.

In a specific embodiment, a membrane is used having both acid groups and base groups, in which case an improvement may be reached in terms of increasing the permeate volume, whilst retention of heavies and ammonium sulphate is at least substantially maintained in case the nanofiltration is carried out with an aqueous liquid having a pH that is above the isoelectric point (pl) of the membrane. Herein, the pl, generally, is the value as provided by the supplier of the membrane.

In accordance with the invention it is also possible to select a specific membrane having a pl below the pH of the aqueous liquid to be treated. It is also possible to adjust the pH of the fluid, by adding a base, e.g. ammonium hydroxide or another hydroxide (for increasing the pH) or an acid (for reducing the pH), e.g. a strong acid, such as sulphuric acid.

The (nanofiltration) membranes used may be selected from hydrophobic and hydrophilic membranes, i.e. membranes of which the outer surface and/or the pore surface are hydrophobic respectively hydrophylic. A hydrophilic membrane is advantageous for a high flux.

The membrane used, and in particular the nanofiltration membrane, may be employed in any suitable form. The membrane configuration may also be selected, e.g. from flat sheet membranes, tubular membranes, and hollow fibres. ‘High shear’ membranes, such as vibrating membranes and rotating membranes can also be used.

A suitable trans-membrane pressure (ΔP), i.e. the pressure difference over the membrane between retentate side and permeate side of the membrane may be chosen within a wide range. The upper limit is in principle determined by the maximally allowable pressure for a specific nanofiltration membrane. In particular, ΔP may be 60 bar or less, 50 bar or less, 40 bar or less, or 35 bar or less. In particular, ΔP may be at least 10 bar, at least 20 bar, at least 25 bar or at least 30 bar.

Preferably, a trans-membrane pressure difference in the range of from 5 to 60 bar is applied in the treatment by means of the membrane process, in particular of from 10 to 50 bar, more in particular of from 15 to 40 bar.

Also with respect to the pH value of the aqueous solution to be subjected to the membrane process of the invention a rather wide range may be chosen without serious negative effects to the results of the process. The pH of the aqueous solution to be subjected to the membrane treatment step is defined as the pH as can be measured by a glass electrode pH meter at room temperature (25° C.). If desired the pH can be adjusted by adding a base or acid to improve performance of the membrane treatment step. The preferred pH depends on the membrane that is used and can be chosen on information available from the membrane supplier, common general knowledge and the information disclosed herein.

Preferably, the pH of the aqueous solution subjected to the treatment by means of the membrane process is in the range of from 1 to 12, in particular in the range of from 4 to 8.

The membrane treatment step according to the invention is usually carried at a temperature between ambient temperature and the so called temperature resistance temperature (such as specified by the supplier). Usually the temperature in the membrane treatment is 95° C. or less. In particular, the membrane treatment, especially the nanofiltration, may be carried out at a temperature of up to 60° C., more in particular at a temperature of up to 50° C. or up to 40° C. Usually, the membrane treatment is carried out at a temperature of at least 5° C. In particular, it may be carried out at a temperature of at least 15° C. In a particularly preferred method, the temperature is in the range of 20 to 60° C. For an advantageous flux, the temperature is preferably at least 25° C., more in particular at least 30° C.

The cross-flow velocity of the liquid through the membrane is usually is at least 1 m/s, in particular at least 2 m/s for a low tendency of fouling and/or a good flux. The cross-flow velocity is usually about 5 m/s or less. Higher velocities may be used, but in general the benefit of additional flux does not outweigh the extra energy consumption at a very high cross-flow velocity. In particular, the cross-flow velocity may be 5 m/s or less.

In suitable embodiments of the process of the invention, and in particular if the membrane in the membrane filtration step is a nanofiltration membrane, the aqueous solution containing caprolactam, ammonium sulphate and one or more other impurities is first subjected to one or more pretreatment steps selected from e.g. pre-filtration to remove solid substances, microfiltration, ultrafiltration, pH adjustment, dilution (e.g. with water) and combinations thereof, before being subjected to the membrane process of the invention.

One or more light compounds may be present in the aqueous solution to be subjected to the membrane treatment. However, in particular hydrophobic organic compounds, such as benzene or another organic solvent which may have been used for caprolactam extraction are preferably removed if the concentration is such that the compound may adversely affect the membrane treatment step, e.g. due to an undesired interaction with the membrane. Suitable methods for removing a hydrophobic organic compound are generally known in the art and include stripping, e.g. benzene stripping for removing benzene. Thus, preferably the aqueous solution containing caprolactam, ammonium sulphate and one or more other impurities is first subjected to removal of organic components having a boiling point lower than that of caprolactam before being treated by means of the membrane process.

In a specific embodiment two or more different nanofiltration membranes are used, which may be used in a series of nanofiltration steps. The nanofiltration membranes may differ, e.g., in that they are made of different materials and/or in that the molecular weight cut-off is different and/or in that they bear different functional groups.

In a specific embodiment method according to the invention, the nanofiltration comprises two or more nanofiltration steps, wherein the permeate of a first nanofiltration step or part thereof is subjected to a further nanofiltration step, thereby forming a further permeate and a further retentate. The further retentate may be discarded or recycled to a previous nanofiltration step. The further permeate or part thereof may in particular be subjected to yet a further nanofiltration step or be recycled to the caprolactam purification process, optionally after another treatment. Usually, the permeate obtained after the last nanofiltration step, or part thereof, is recycled into the purification process for the caprolactam streams. The use of more than one nanofiltration step may in particular be advantageous with respect to a high caprolactam content in the permeate relative to the amounts of ammonium sulphate and heavies, in particular sulphonated components.

Accordingly, preferably the treatment by means of the membrane process is carried out in at least two subsequent membrane treatment steps, wherein at least part of the permeate of the first membrane treatment step is fed to a second membrane treatment step, wherein more than 60 wt. % of the caprolactam in the permeate solution of the first membrane step is passed through the second membrane to the second permeate side, thereby forming a purified caprolactam containing second permeate and a second retentate, wherein the second retentate may be discarded or recycled to the first membrane treatment step and wherein the further permeate stream optionally may be subjected to one or more further membrane treatment steps to obtain further retentate(s) and permeate(s).

Any of the plurality of membranes used if more than one nanofiltration step is employed may be the same or different and/or operated under the same of different conditions.

For instance, in an advantageous method making use of at least two nanofiltration steps, a first step is a nanofiltration with a relatively high aqueous flux through the membrane in the first step compared to the flux through the membrane in the second nanofiltration step and a second is a nanofiltration with a relatively high rejection of impurities through the membrane in the second step compared to the membrane in the first step. It is contemplated that in total thus less membrane area is required.

The present invention allows recovery of purified caprolactam from aqueous streams containing caprolactam, which otherwise would have been considered as waste streams. In particular, the caprolactam is recovered from the purified caprolactam containing permeate and/or second or further permeate stream(s).

The invention will now be illustrated by the following examples.

Experimental Part

Analytical Methods

The following analytical techniques were used:
a) Caprolactam: Analysis of caprolactam was performed by gas chromatography.
b) (NH₄)₂SO₄: Analysis of ammonium sulphate (AS) was done titrimetrically, after stripping with nitrogen gas at 60° C.
c) Total sulphur: Total sulphur concentration was determined after a microwave destruction of the sample with aqua regia, inductively coupled plasma-atomic emission spectrometry using (ICP-AES).
d) Organic sulphur content: The content of organic sulphur (from sulphonated organics) is then calculated by subtracting the sulphur content as present in AS from the total sulphur content.

### EXAMPLE 1

600 g of eluate waste water comprising 1.5 wt. % caprolactam, 3.0 wt. % ammonium sulphate, 1.5 wt. % heavies (mainly sulphonated caprolactam) and traces of organic lights, were introduced into the thermostated feed tank of a batchwise operated cross-flow filtration unit. This aqueous stream was obtained after extraction with benzene of an aqueous caprolactam stream after Beckmann rearrangement and neutralisation by adding concentrated base. The pH level of the feed was increased to 5. The unit was equipped with a polyamide XN45 nanofiltration membrane (Supplier Trisep; polyamide; cut-off of 200 g/mol; pl=neutral; water flux at 12 bar and 18° C. of 174 kg/hr/m²). The vessel was closed and the circulation pump started allowing for a cross flow velocity of 3.5 m/s. A constant pressure of 30 bar was applied on the feed side of the membrane by means of nitrogen gas and the temperature was set to 30° C. The permeate flow was monitored over the filtration time and the flux was determined by weight difference. Various samples of permeate as well as concentrate were taken at different time intervals and analyzed in terms of caprolactam, ammonium sulphate and sulphur concentration.

The experiment was also repeated with another membrane, namely a TFC SR2 membrane (supplier Koch, Germany; cut off: 400 g/mol; polyamide; pl: pH=3; water flux at 30 bar and 20° C. of 450 kg/hr/m²)

The results of the nanofiltration tests are presented in Table 1 for these membranes. At a concentration factor of 4 (i.e. concentration in feed divided by concentration in permeate=4), the fluxes for XN45 and TFC SR2 were about 10 kg/hr/m².

In Table 1, the caprolactam, ammonium sulphate and total sulphur concentrations in the initial feed and in the permeate (at a concentration factor of 4) are presented. As can be seen, none of these membranes showed substantial retention for caprolactam, thus more than 50 wt. % of the caprolactam permeated through the membrane allowing for a good recovery.

TABLE 1
concentrations in the initial feed and in the permeate

Concentration (wt. %)

Permeate

| Component | Feed | XN45 | TFC SR2 |
|---|---|---|---|
| Caprolactam | 1.35 | 1.45 | 1.47 |
| Ammonium sulphate | 3.80 | 1.30 | 0.91 |
| Organic sulphur content | 1.20 | 0.57 | 0.42 |

### EXAMPLE 2

Similar experiments as in the example 1 were performed using the Trisep XN45 membrane, but now using a second membrane treatment step according to the invention. The permeate obtained in the first filtration stage (as obtained in example 1) was used as feed for the second stage. High fluxes (30 kg/hr/m²) were obtained even at a volumetric concentration factor as high as 8. Table 2 presents the analysed concentrations of caprolactam, ammonium sulphate and organic sulphur in the initial feed and in the 1^{st} and 2^{nd} stage permeates. An improvement in the permeate purity was obtained with about 9 times lower ammonium sulphate and 6 times lower organic sulphur concentrations in the second stage permeate. About 70% of the initial amount of caprolactam was recovered in a two stage nanofiltration process while rejecting 90% of ammonium sulphate and over 80% of the sulphonated organic components. The purified caprolactam stream (permeate stream) could be reintegrated into the caprolactam purification part of the main stream of the caprolactam process.

TABLE 2
concentrations in the initial feed and
in the 1^{st} and 2^{nd} stage permeates

Concentration (wt. %)

Permeate

| Component | Feed | 1^{st} stage | 2^{nd} stage |
|---|---|---|---|
| Caprolactam | 1.35 | 1.45 | 1.47 |
| Ammonium sulphate | 3.80 | 1.30 | 0.40 |
| Organic sulphur content | 1.20 | 0.57 | 0.20 |

1. Process for treating an aqueous solution containing caprolactam, ammonium sulphate and one or more other impurities, comprising one or more organic impurities from a caprolactam production process and optionally other salts than ammonium sulphate, by means of a membrane process, thereby obtaining a retentate and a permeate,
characterized in that
the membrane used is selected from the group of polyether sulphone membranes, sulphonated polyether sulphone membranes, polyester membranes, polysulphone membranes, aromatic polyamide membranes, polyvinyl alcohol membranes, polypiperazine membranes, cellulose acetate membranes, titanium oxide membranes, zirconium oxide membranes and aluminium oxide membranes, having a molecular weight cut off in the range of 100-1000 g/mol;
and in that more than 60 wt. % of the caprolactam in the aqueous solution is passed through a membrane to the permeate side, to obtain a purified caprolactam containing permeate stream,
and in that at least 50 wt. % of the organic impurities are retained in the retentate solution.

2. Process according to claim 1, characterized in that the molecular weight cut off of the membrane used is in the range of 150-400 g/mol.

3. Process according to claim 1, characterized in that at least 50 wt. % of the ammonium sulphate in the aqueous solution containing caprolactam, ammonium sulphate and one or more other impurities is retained in the retentate solution.

4. Process according to claim 1, characterized in that the aqueous solution containing caprolactam, ammonium sulphate and one or more other impurities is a solution from a caprolactam production process, and that at least part of the permeate solution stream is re-used in a caprolactam production process, or in a section of another process with streams containing caprolactam.

5. Process according to claim 4, characterized in that the caprolactam process is a hydroxylammonium sulphate or phosphate oxime process with formation of the oxime from cyclohexanone via an oximation process followed by Beckmann rearrangement of the oxime, neutralisation with aqueous ammonia and purification of the caprolacta obtained.

6. Process according to claim 1, characterized in that at least 80 wt. %, of the caprolactam in the aqueous solution containing caprolactam, ammonium sulphate and one or more other impurities is passed through the membrane to the permeate side.

7. Process according to claim 1, characterized in that the caprolactam concentration in the permeate stream is higher than the caprolactam concentration in the aqueous solution that is subjected to the membrane treatment.

8. Process according to claim 1, characterized in that at least 60 wt. %, of the organic impurities in the aqueous solution containing caprolactam, ammonium sulphate and one or more other impurities are retained in the retentate solution.

9. Process according to claim 1, characterized in that the total concentration of organic impurities in the permeate is at least 1.5 times lower than their total concentration in the aqueous solution before the treatment by means of the membrane process.

10. Process according to claim 1, characterized in that a trans-membrane pressure difference in the range of from 5 to 60 bar is applied in the treatment by means of the membrane process.

11. Process according to claim 1, characterized in that the pH of the aqueous solution subjected to the treatment by means of the membrane process is in the range of from 1 to 12.

12. Process according to claim 11, characterized in that the pH is in the range of from 4 to 8.

13. Process according to claim 1 characterized in that the aqueous solution containing caprolactam, ammonium sulphate and one or more other impurities is first subjected to one or more pretreatment steps selected from pre-filtration to remove solid substances, microfiltration, ultrafiltration, pH adjustment, dilution and combinations thereof, before being subjected to the membrane process of claim 1.

14. Process according to claim 1, characterized in that the aqueous solution containing caprolactam, ammonium sulphate and one or more other impurities is first subjected to removal of organic components having a boiling point lower than that of caprolactam before being treated by means of the membrane process.

15. Process according to claim 1, characterized in that the treatment by means of the membrane process is carried out in at least two subsequent membrane treatment steps, wherein at least part of the permeate of the first membrane treatment step is fed to a second membrane treatment step, wherein more than 60 wt. % of the caprolactam in the permeate solution of the first membrane step is passed through the second membrane to the second permeate side, thereby forming a purified caprolactam containing second permeate and a second retentate, wherein the second retentate may be discarded or recycled to the first membrane treatment step and wherein the further permeate stream optionally may be subjected to one or more further membrane treatment steps to obtain further retentate(s) and permeate(s).

16. Process according to claim 1, characterized in that caprolactam is recovered from the purified caprolactam containing permeate and/or second or further permeate stream(s).
